(19) United States
(12) Patent Application Publication
(10) Pub. No.: US 2024/0172920 A1
(43) Pub. Date: May 30, 2024

(54) ENDOSCOPE DEVICE, METHOD OF OPERATING ENDOSCOPE DEVICE, AND RECORDING MEDIUM

(71) Applicant: Evident Corporation, Nagano (JP)

(72) Inventor: Hideyoshi YAMAUCHI, Akiruno-shi (JP)

(73) Assignee: Evident Corporation, Nagano (JP)

(21) Appl. No.: 18/385,536

(22) Filed: Oct. 31, 2023

(30) Foreign Application Priority Data
Nov. 25, 2022 (JP) 2022-188390

(51) Int. Cl. A61B 1/00; H04N 23/73; H04N 23/74

(52) U.S. Cl. CPC A61B 1/000095; A61B 1/00006; H04N 23/73; H04N 23/74

(57) ABSTRACT

An endoscope device includes an imaging device, a determination unit, and a control unit. The determination unit is configured to determine the position and the size of a region including the distal end of a treatment tool seen in an image. The control unit is configured to set a control region in the image based on the position and the size. The control unit is configured to execute exposure control of controlling brightness of the image by using a value of a pixel in the control region. The control unit is configured to display the control region on a display. The control region includes a first region and excludes a second region. The distal end is seen in the first region. Abase end of the treatment tool is seen in the second region. The second region is in contact with a boundary of the image.

### BACKGROUND OF THE INVENTION

### Field of the Invention

The present invention relates to an endoscope device, a method of operating the endoscope device, and a recording medium.

Priority is claimed on Japanese Patent Application No. 2022-188390, filed on Nov. 25, 2022, the content of which is incorporated herein by reference.

### Description of Related Art

Industrial endoscope devices have been used for inspection of internal damage, corrosion, and the like of boilers, pipes, aircraft engines, and the like. An endoscope device includes an insertion unit to be inserted into an inspection target. The insertion unit acquires an image of a subject in the inspection target.

In some cases, a treatment tool is used for, for example, collecting a foreign object attached on the surface of a subject. While the treatment tool is used, the distal end and base end (root) of the treatment tool are seen in an image. Halation may occur at the base end of the treatment tool and the visibility of the image may deteriorate. In a case in which the visibility of the image deteriorates, a user has difficulty in operating the treatment tool.

A technique disclosed in Japanese Unexamined Patent Application, First Publication No. 2012-120618 provides a method of improving the visibility of images. The technique determines whether a treatment tool is included in an imaging range of an imaging unit. When the treatment tool is included in the imaging range, the technique adjusts a diaphragm of the imaging unit. By doing this, the technique prevents deterioration of the quality of an image due to the blurring of the image. In addition, the technique increases the brightness of the image after the diaphragm is adjusted.

### SUMMARY OF THE INVENTION

According to a first aspect of the present invention, an endoscope device includes an imaging device, a determination unit, and a control unit. The imaging device is configured to generate an image in which a treatment tool is seen. The determination unit is configured to determine the position and the size of a region including the distal end of the treatment tool seen in the image. The control unit is configured to: set a control region in the image based on the position and the size determined by the determination unit; execute exposure control of controlling the brightness of the image by using a value of a pixel in the control region; and display, on a display, a control region image including the control region after the exposure control is executed. The control region includes a first region and excludes a second region. The distal end is seen in the first region. The base end of the treatment tool is seen in the second region. The second region is in contact with the boundary of the image.

According to a second aspect of the present invention, in the first aspect, the control unit may be configured to: determine whether halation has occurred in the image; and execute the exposure control when the control unit has determined that the halation has occurred.

According to a third aspect of the present invention, in the second aspect, the control unit may be configured to set the control region that excludes a region in which the halation has occurred when the control unit has determined that the halation has occurred.

According to a fourth aspect of the present invention, in the second aspect, the endoscope device may further include an image-processing circuit configured to adjust the brightness of the image by using a gain value. The control unit may be configured to execute the exposure control by controlling the gain value used for adjusting the brightness of the control region when the control unit has determined that the halation has occurred.

According to a fifth aspect of the present invention, in the second aspect, the control unit may be configured to execute the exposure control by using a value of a pixel in a region that includes the control region and is larger than the control region when the control unit has determined that the halation has not occurred.

According to a sixth aspect of the present invention, in the fifth aspect, the control unit may be configured to execute the exposure control by controlling at least one of an amount of illumination light emitted to the treatment tool and an exposure amount of the imaging device when the control unit has determined that the halation has not occurred.

According to a seventh aspect of the present invention, in the first aspect, the determination unit may be configured to: learn the shape of the treatment tool by using the image; generate learning data indicating the features of the shape; and determine the position and the size by using the learning data.

According to an eighth aspect of the present invention, in the first aspect, the imaging device may be disposed in the distal end portion of an insertion unit to be inserted inside an object. An optical adaptor including a lens may be connected to the distal end portion. The control unit may be configured to: determine the distance from the distal end portion to the distal end of the treatment tool by using the value of the pixel; and execute the exposure control based on the distance.

According to a ninth aspect of the present invention, in the eighth aspect, the control unit may be configured to determine the distance based on the brightness of the treatment tool indicated by the value of the pixel.

According to a tenth aspect of the present invention, in the eighth aspect, the control unit may be configured to: determine a type of the optical adaptor; acquire, from a memory, distance information including a type of the optical adaptor, the size of the treatment tool, and the distance; and determine the distance based on the type determined by the control unit, the size determined by the determination unit, and the distance information.

According to an eleventh aspect of the present invention, in the first aspect, the control unit may be configured to: generate a partial image by cutting out the control region from the image generated by the imaging device; and display the image generated by the imaging device and the partial image on the display such that at least part of the image generated by the imaging device and at least part of the partial image overlap each other.

According to a twelfth aspect of the present invention, in the first aspect, the control unit may be configured to: generate a partial image by cutting out the control region from the image generated by the imaging device; and display the image generated by the imaging device and the partial image on the display such that the image generated by the imaging device and the partial image do not overlap each other.

According to a thirteenth aspect of the present invention, in the first aspect, the control unit may be configured to: generate a partial image by cutting out the control region from the image generated by the imaging device; and display an image generated by enlarging the partial image on the display.

According to a fourteenth aspect of the present invention, in the first aspect, the control unit may be configured to: determine a degree of blurring of the distal end by using the value of the pixel; and perform edge enhancement on the image based on the degree.

According to a fifteenth aspect of the present invention, a method of operating an endoscope device including an imaging device configured to generate an image in which a treatment tool is seen is provided. The method includes: determining the position and the size of a region including the distal end of the treatment tool seen in the image; setting a control region in the image based on the position and the size; executing exposure control of controlling the brightness of the image by using a value of a pixel in the control region; and displaying, on a display, a control region image including the control region after the exposure control is executed. The control region includes a first region and excludes a second region. The distal end is seen in the first region. The base end of the treatment tool is seen in the second region. The second region is in contact with the boundary of the image.

According to a sixteenth aspect of the present invention, a non-transitory computer-readable recording medium stores a program causing a computer of an endoscope device including an imaging device configured to generate an image in which a treatment tool is seen to execute: determining the position and the size of a region including the distal end of the treatment tool seen in the image; setting a control region in the image based on the position and the size; executing exposure control of controlling the brightness of the image by using a value of a pixel in the control region; and displaying, on a display, a control region image including the control region after the exposure control is executed. The control region includes a first region and excludes a second region. The distal end is seen in the first region. The base end of the treatment tool is seen in the second region. The second region is in contact with the boundary of the image.

According to a seventeenth aspect of the present invention, an endoscope device includes an imaging device and a processor. The imaging device is configured to generate an image in which a treatment tool is seen. The processor is configured to: determine the position and the size of a region including the distal end of the treatment tool seen in the image; set a control region in the image based on the position and the size; execute exposure control of controlling the brightness of the image by using a value of a pixel in the control region; and display, on a display, a control region image including the control region after the exposure control is executed. The control region includes a first region and excludes a second region. The distal end is seen in the first region. The base end of the treatment tool is seen in the second region. The second region is in contact with the boundary of the image.

### BRIEF DESCRIPTION OF THE DRAWINGS

FIG. 1 is a block diagram showing a configuration of an endoscope device according to a first embodiment of the present invention.

FIG. 2 is a block diagram showing a configuration of an insertion unit and an optical adaptor in the first embodiment of the present invention.

FIG. 3 is a block diagram showing an example of a treatment tool in the first embodiment of the present invention.

FIG. 4 is a block diagram showing an example of a treatment tool in the first embodiment of the present invention.

FIG. 5 is a flow chart showing a procedure of image display processing executed by the endoscope device according to the first embodiment of the present invention.

FIG. 6 is a diagram showing an example of an image in the first embodiment of the present invention.

FIG. 7 is a diagram showing an example of an image in the first embodiment of the present invention.

FIG. 8 is a block diagram showing an example of a partial image in the first embodiment of the present invention.

FIG. 9 is a diagram showing an example of an image in the first embodiment of the present invention.

FIG. 10 is a diagram showing an example of an image in the first embodiment of the present invention.

FIG. 11 is a flow chart showing a procedure of image display processing executed by an endoscope device according to a second embodiment of the present invention.

FIG. 12 is a graph showing an example of resolution characteristic information in the second embodiment of the present invention.

FIG. 13 is a flow chart showing a procedure of image display processing executed by an endoscope device according to a third embodiment of the present invention.

FIG. 14 is a flow chart showing a procedure of image display processing executed by an endoscope device according to a fourth embodiment of the present invention.

FIG. 15 is a flow chart showing a procedure of image display processing executed by an endoscope device according to a fifth embodiment of the present invention.

### DETAILED DESCRIPTON OF THE INVENTION

Hereinafter, embodiments of the present invention will be described with reference to the drawings.

### First Embodiment

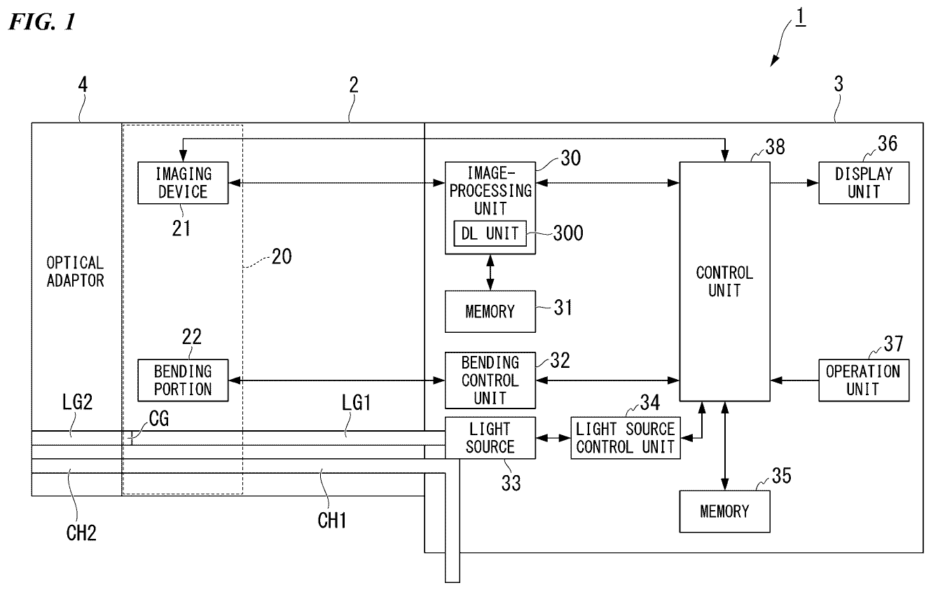

A first embodiment of the present invention will be described. FIG. 1 shows a configuration of an endoscope device 1 according to the first embodiment. The endoscope device 1 shown in FIG. 1 includes an insertion unit 2 and a main body unit 3.

The insertion unit 2 constitutes an endoscope. The insertion unit 2 is inserted into the inside of an inspection target. A distal end portion 20 is disposed in the distal end of the insertion unit 2. The insertion unit 2 has a long and thin bendable tube shape from the distal end portion 20 to a base end portion. An optical adaptor 4 is mounted on the distal end portion 20. The main body unit 3 is a control device including a housing unit that houses the insertion unit 2.

The insertion unit 2 includes an imaging device 21 and a bending portion 22. The main body unit 3 includes an image-processing unit 30, a memory 31, a bending control unit 32, a light source 33, a light source control unit 34, a memory 35, a display unit 36, an operation unit 37, and a control unit 38.

A schematic configuration of the endoscope device 1 will be described. The imaging device 21 generates an image in which a treatment tool is seen. The light source 33 generates illumination light. The image-processing unit 30 (determination unit) determines the position and the size of a region including the distal end of the treatment tool seen in the image generated by the imaging device 21. The control unit 38 sets a control region in the image generated by the imaging device 21 based on the position and the size determined by the image-processing unit 30. The control unit 38 executes exposure control of controlling the brightness of the image by using a value of a pixel in the control region. The control unit 38 displays the control region on the display unit 36. The control region includes a first region and excludes a second region. The distal end of the treatment tool is seen in the first region. The base end of the treatment tool is seen in the second region. The second region is in contact with the boundary of the image.

A detailed configuration of the endoscope device 1 will be described. The imaging device 21 and the bending portion 22 are disposed in the distal end portion 20.

The imaging device 21 is an image sensor such as a CCD sensor or a CMOS sensor. The imaging device 21 sequentially generates two or more images (frames). The two or more images constitute a video. The imaging device 21 outputs each of the generated images to the main body unit 3.

The imaging device 21 includes two or more pixels. Each pixel generates an analog signal in accordance with the amount of light incident on each pixel. The image generated by the imaging device 21 has values of two or more pixels corresponding to the two or more pixels in the imaging device 21. For example, the imaging device 21 outputs an image (analog image) including the analog signal generated in each pixel. The image is converted into a digital image by an analog-to-digital (AD) converter not shown in FIG. 1. Alternatively, the imaging device 21 includes an AD converter and outputs a digital image processed by the AD converter.

The bending portion 22 is disposed on the base end side in the distal end portion 20. The bending portion 22 bends the distal end portion 20 in a predetermined direction.

The image-processing unit 30 (image-processing circuit) processes the image output from the imaging device 21. The image-processing unit 30 includes a DL unit 300. The DL unit 300 executes machine learning that uses the image output from the imaging device 21 and learns the shape of a treatment tool seen in the image. For example, the DL unit 300 executes deep learning. The DL unit 300 generates learning data indicating the features of the shape of the treatment tool. Thereafter, the DL unit 300 determines the position and the size of the treatment tool in the image by using the learning data. The DL unit 300 outputs the processed image to the control unit 38. In addition, the DL unit 300 outputs position information indicating the position of the treatment tool and size information indicating the size of the treatment tool to the control unit 38.

The analog image output from the imaging device 21 is converted into a digital image by an AD converter not shown in FIG. 1. Alternatively, the imaging device 21 outputs the digital image. The image-processing unit 30 may adjust the brightness of the digital image by using a gain value. Specifically, the image-processing unit 30 may adjust a value of each pixel of the image by multiplying the value of each pixel by the gain value.

The memory 31 is a volatile or nonvolatile memory. For example, the memory 31 is at least one of a random-access memory (RAM), a dynamic random-access memory (DRAM), a static random-access memory (SRAM), an erasable programmable read-only memory (EPROM), an electrically erasable programmable read-only memory (EEPROM), and a flash memory. The memory 31 stores the image output from the imaging device 21 and the learning data generated by the image-processing unit 30.

The bending control unit 32 bends the bending portion 22 based on the control signal output from the control unit 38.

The light source 33 is a light-emitting device such as a light-emitting diode (LED). The light source 33 generates illumination light. The light source 33 may be disposed in the distal end portion 20. The light source control unit 34 controls the amount of light of the light source 33 based on the control signal output from the control unit 38.

The memory 35 is a volatile or nonvolatile memory. For example, the memory 35 is at least one of a RAM, a DRAM, an SRAM, an EPROM, an EEPROM, and a flash memory. The memory 35 stores data and information processed by the control unit 38. The memory 31 and the memory 35 may be integrated.

The display unit 36 is a monitor (display) such as a liquid crystal display (LCD). The display unit 36 may be a touch panel. In a case in which the display unit 36 is configured as a touch panel, a user touches the display screen of the display unit 36 by using a part of the body or a tool. For example, the part of the body is a finger. The display unit 36 displays the image generated by the imaging device 21.

The operation unit 37 includes a button, a joystick, and the like for a user to perform an operation. The user inputs various kinds of information into the endoscope device 1 by operating the operation unit 37. For example, the various kinds of information include a bending instruction to bend the distal end portion 20.

The control unit 38 controls the entire endoscope device 1. For example, the control unit 38 executes the following processing.

The control unit 38 receives the image, the position information, and the size information from the image-processing unit 30. The control unit 38 sets a control region in the image based on the position information and the size information. The control region includes two or more pixels in the image. The control unit 38 executes the exposure control by using values of one or more pixels in the control region. In other words, the control unit 38 executes the exposure control by using all or part of the control region.

The control unit 38 executes the exposure control by controlling the amount (brightness) of the illumination light in the light source 33. Specifically, the control unit 38 generates a control signal used for controlling the amount of the illumination light and outputs the control signal to the light source control unit 34. The light source control unit 34 controls the amount of light of the light source 33.

Alternatively, the control unit 38 executes the exposure control by controlling the exposure amount of the imaging device 21. Specifically, the control unit 38 generates a control signal used for controlling the exposure amount and outputs the control signal to the imaging device 21. The imaging device 21 controls the exposure amount based on the control signal.

For example, the imaging device 21 controls an exposure time (shutter speed) or the sensitivity, thus controlling the exposure amount. The imaging device 21 controls a start timing of the exposure and a completion timing of the exposure, thus controlling the exposure time. An analog signal is generated in accordance with the amount of light incident on each pixel. Generation of the analog signal is started at the start timing. The generation of the analog signal is completed at the completion timing. The imaging device 21 controls a gain value by which the analog signal generated in each pixel is multiplied, thus controlling the sensitivity.

The control unit 38 controls each unit of the endoscope device 1 based on various kinds of information input via the operation unit 37. For example, the control unit 38 generates a control signal used for controlling a bending operation of the bending portion 22 based on the bending instruction and outputs the control signal to the bending control unit 32.

At least one of the image-processing unit 30, the bending control unit 32, the light source control unit 34, and the control unit 38 may be constituted by at least one of a processor and a logic circuit. For example, the processor is at least one of a central processing unit (CPU), a digital signal processor (DSP), and a graphics-processing unit (GPU). For example, the logic circuit is at least one of an application-specific integrated circuit (ASIC) and a field-programmable gate array (FPGA). At least one of the image-processing unit 30, the bending control unit 32, the light source control unit 34, and the control unit 38 may include one or a plurality of processors. At least one of the image-processing unit 30, the bending control unit 32, the light source control unit 34, and the control unit 38 may include one or a plurality of logic circuits.

A computer of the endoscope device 1 may read a program and execute the read program. The program includes commands defining the operations of each unit of the endoscope device 1. In other words, the functions of the endoscope device 1 may be realized by software.

The program described above, for example, may be provided by using a “computer-readable storage medium” such as a flash memory. The program may be transmitted from the computer storing the program to the endoscope device 1 through a transmission medium or transmission waves in a transmission medium. The “transmission medium” transmitting the program is a medium having a function of transmitting information. The medium having the function of transmitting information includes a network (communication network) such as the Internet and a communication circuit line (communication line) such as a telephone line. The program described above may realize some of the functions described above. In addition, the program described above may be a differential file (differential program). The functions described above may be realized by a combination of a program that has already been recorded in a computer and a differential program.

A light guide LG1 is disposed inside the insertion unit 2 and the main body unit 3. The light guide LG1 transmits the illumination light generated by the light source 33 to the distal end portion 20. A transparent cover glass CG is disposed in the distal end of the insertion unit 2. The light guide LG1 is connected to the cover glass CG. The illumination light passes through the light guide LG1 and the cover glass CG and is incident on a light guide LG2 disposed inside the optical adaptor 4. The illumination light passes through the light guide LG2 and is emitted to a subject.

A channel CH1 is disposed inside the insertion unit 2 and the main body unit 3. A treatment tool is inserted into the channel CH1. The treatment tool passes through the channel CH1 and is inserted into a channel CH2 disposed inside the optical adaptor 4. The treatment tool passes through the channel CH2 and protrudes ahead of the optical adaptor 4. The distal end of the treatment tool has a shape in accordance with the type of the treatment tool.

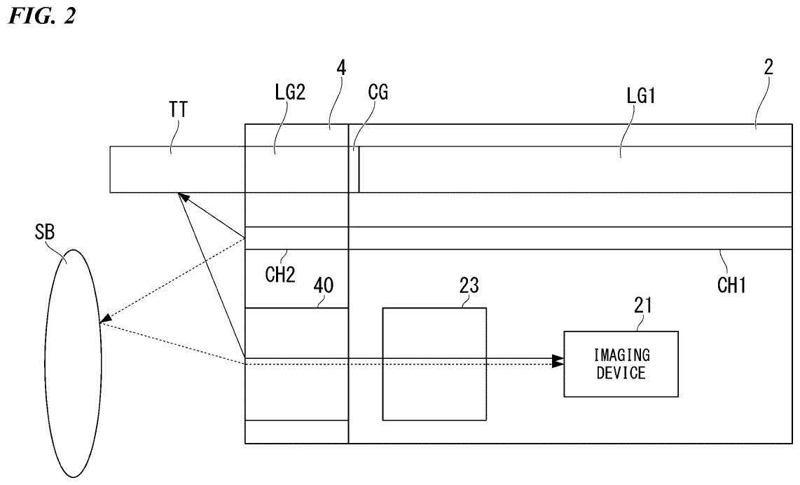

FIG. 2 shows a detailed configuration of the insertion unit 2 and the optical adaptor 4. A lens 23 is disposed in the insertion unit 2, and a lens 40 is disposed in the optical adaptor 4.

A treatment tool TT is inserted into the channel CH1 and the channel CH2. The treatment tool TT protrudes from the front surface of the optical adaptor 4.

The illumination light generated by the light source 33 passes through the light guide LG1, the cover glass CG, and the light guide LG2 and is emitted to a subject SB. Part of the illumination light is also emitted to the treatment tool TT. The illumination light is reflected by the surface of the subject SB and is incident on the lens 40. In addition, the illumination light is reflected by the surface of the treatment tool TT and is incident on the lens 40. The light incident on the lens 40 passes through the lens 40 and is incident on the lens 23. The light incident on the lens 23 reaches the imaging device 21.

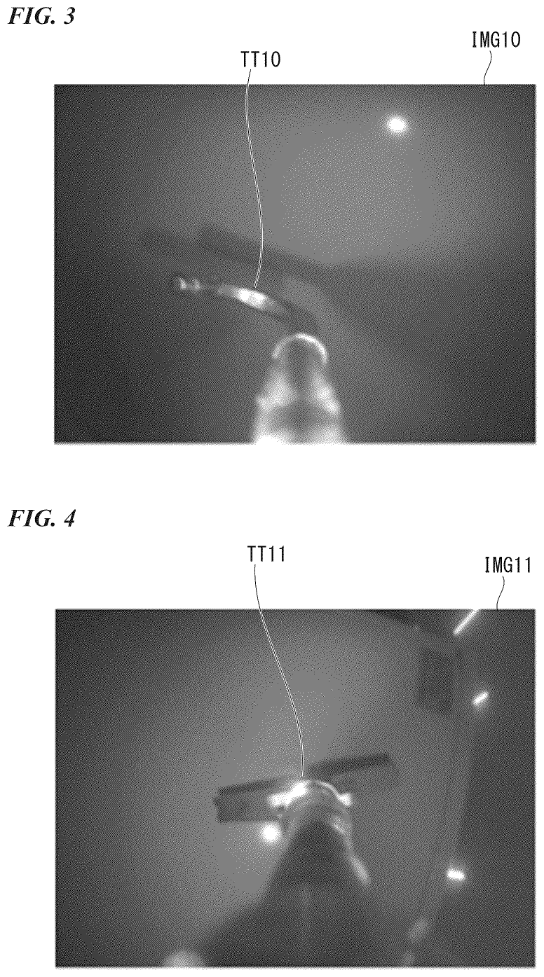

FIG. 3 and FIG. 4 show examples of a treatment tool. A treatment tool TT10 is seen in an image IMG10 shown in FIG. 3. The distal end of the treatment tool TT10 is to be hung on the edge of an inspection portion in an inspection target. A treatment tool TT11 is seen in an image IMG11 shown in FIG. 4. The treatment tool TT11 captures a foreign object in the inspection target. A treatment tool used in the endoscope device 1 is not limited to the treatment tool TT10 and the treatment tool TT11.

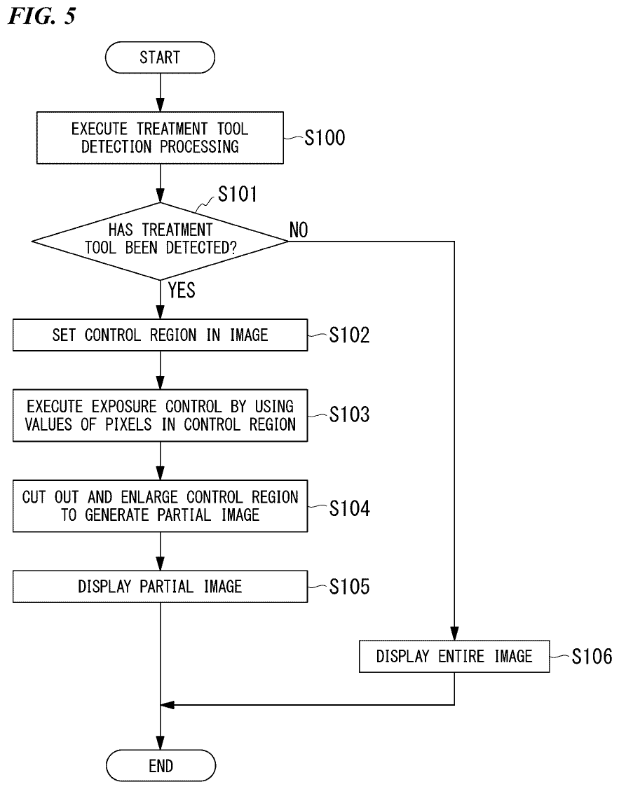

The endoscope device 1 executes image display processing shown in FIG. 5. FIG. 5 shows a procedure of the image display processing.

The DL unit 300 executes treatment tool detection processing by using the image output from the imaging device 21 (Step S100).

The DL unit 300 executes the following processing in Step S100. The DL unit 300 reads the learning data from the memory 31. The DL unit 300 executes deep learning that uses the learning data and detects a region having similar features to those of a treatment tool indicated by the learning data. In other words, the DL unit 300 detects a region in the image in which the treatment tool is seen.

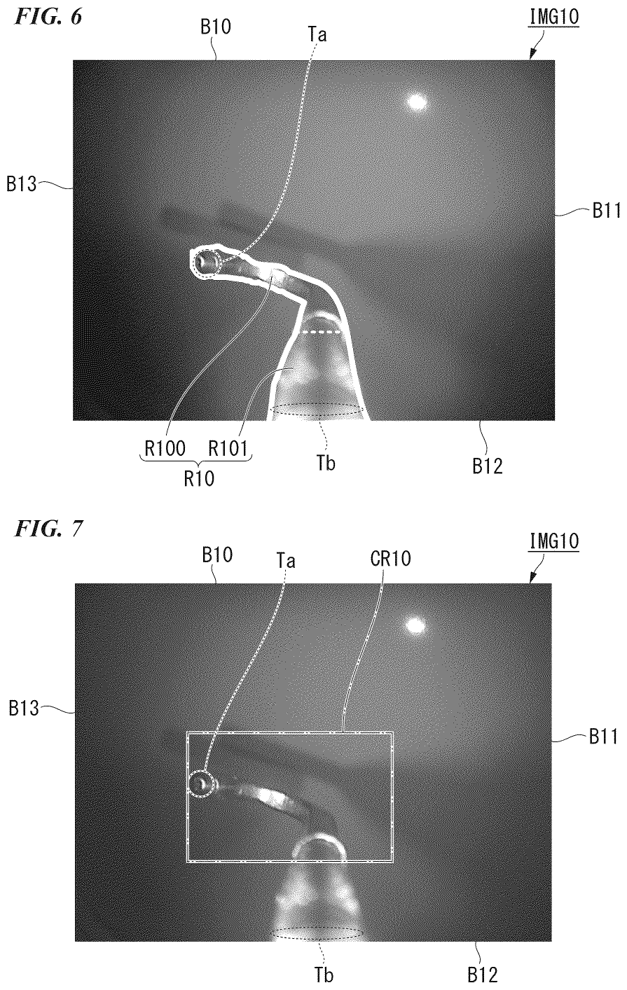

FIG. 6 is an example of the image generated by the imaging device 21. An image IMG10 is shown in FIG. 6. The image IMG10 shown in FIG. 6 is the same as that shown in FIG. 3. The DL unit 300 detects a region R10 in which a treatment tool is seen in the image IMG10.

A positional relationship between the imaging device 21 and the channel CH1 is fixed. Therefore, a region in which a treatment tool is seen in the image IMG10 is fixed. The image IMG10 is rectangular and has four boundaries B10, B11, B12, and B13. The boundaries B10, B11, B12, and B13 are on the outer periphery of the image IMG10.

A treatment tool seen in the image IMG10 extends from the boundary B12 toward the center of the image IMG10. A distal end Ta of the treatment tool is at a position close to the center of the image IMG10. The distal end Ta of the treatment tool is seen at a position that is in accordance with the amount by which the treatment tool is pulled out of the distal end of the insertion unit 2. The distance between the distal end of the insertion unit 2 and the distal end Ta of the treatment tool increases as the amount increases. The distal end Ta of the treatment tool nears the center of the image IMG10 as the amount increases.

A base end Tb of the treatment tool meets the boundary B12. The base end Tb of the treatment tool is a portion of the treatment tool that is farthest away from the center of the image IMG10.

The region R10 includes a first region R100 and a second region R101. The distal end Ta of the treatment tool is seen in the first region R100. The second region R101 meets the boundary B12. The base end Tb of the treatment tool is seen in the second region R101. Halation may occur in the second region R101.

The DL unit 300 determines the position and the size of a region in which the treatment tool is seen and generates position information and size information. The position information indicates the position of the treatment tool. The size information indicates the size of the treatment tool. For example, the position information and the size information are integrated and indicate positions of two or more pixels included in the region R10. The position information may indicate the center position or the centroid position of the region R10. The size information may indicate the number of pixels included in the region R10.

The DL unit 300 outputs the position information and the size information to the control unit 38. In a case in which a region in which the treatment tool is seen has not been detected, the DL unit 300 outputs information indicating that the treatment tool has not been detected to the control unit 38. In addition, the image-processing unit 30 outputs the image generated by the imaging device 21 to the control unit 38.

After Step S100, the control unit 38 determines whether a treatment tool has been detected based on the information output from the DL unit 300 (Step S101). When the position information and the size information are output from the DL unit 300, the control unit 38 determines that the treatment tool has been detected. When the information indicating that the treatment tool has not been detected is output from the DL unit 300, the control unit 38 determines that the treatment tool has not been detected.

When the control unit 38 has determined that the treatment tool has been detected in Step S101, the control unit 38 sets a control region in the image output from the image-processing unit 30. At this time, the control unit 38 sets the control region in the image based on the position information and the size information (Step S102).

FIG. 7 is an example of the image generated by the imaging device 21. An image IMG10 is shown in FIG. 7. The image IMG10 shown in FIG. 7 is the same as that shown in FIG. 3. The control unit 38 sets a control region CR10 shown in FIG. 7 in the image IMG10.

The control region CR10 includes the first region R100 shown in FIG. 6 and excludes the second region R101 shown in FIG. 6. In the example shown in FIG. 7, the control region CR10 is a rectangular region. The shape of the control region CR10 is not limited to a rectangle.

The control region CR10 is part of the image IMG10. The control region CR10 includes part of a region (the first region R100 shown in FIG. 6) in which the treatment tool is seen. Specifically, the control region CR10 includes a region (the first region R100 shown in FIG. 6) in which the distal end Ta of the treatment tool is seen. Accordingly, the control region CR10 is an image of the distal end Ta of the treatment tool.

The control region CR10 excludes a region (the second region R101 shown in FIG. 6) in which at least part of a region other than the distal end Ta of the treatment tool is seen. In other words, the control region CR10 excludes a region in which the base end Tb of the treatment tool is seen. The image IMG10 has boundaries B10, B11, B12, and B13. The control region CR10 excludes the boundaries B10, B11, B12, or B13. The control region CR10 may include a region in which the treatment tool is not seen. The region may include at least one of the boundaries B10, B11, and B13 other than the boundary B12 that meets the base end Tb of the treatment tool.

After Step S102, the control unit 38 executes the exposure control by using values of pixels of the control region set in Step S102 (Step S103).

The control unit 38 executes the following processing in Step S103. For example, the control unit 38 determines the brightness of the control region CR10 shown in FIG. 7 based on the values of the pixels in the control region CR10. The control unit 38 calculates a setting value of the amount of light of the light source 33 based on the brightness. When the brightness of the control region CR10 is lower than predetermined brightness, the control unit 38 calculates a setting value to increase the amount of light of the light source 33. When the brightness of the control region CR10 is higher than the predetermined brightness, the control unit 38 calculates a setting value to reduce the amount of light of the light source 33.

The control unit 38 generates a control signal indicating the calculated setting value and outputs the control signal to the light source control unit 34. The light source control unit 34 controls the amount of light of the light source 33 based on the control signal output from the control unit 38.

Alternatively, the control unit 38 calculates an exposure time of the imaging device 21 based on the brightness of the control region CR10. When the brightness of the control region CR10 is lower than the predetermined brightness, the control unit 38 calculates a longer exposure time than the present exposure time of the imaging device 21. When the brightness of the control region CR10 is higher than the predetermined brightness, the control unit 38 calculates a shorter exposure time than the present exposure time of the imaging device 21.

The control unit 38 generates a control signal indicating the calculated exposure time and outputs the control signal to the imaging device 21. The imaging device 21 controls the exposure time based on the control signal output from the control unit 38.

Alternatively, the control unit 38 calculates the sensitivity of the imaging device 21 based on the brightness of the control region CR10. When the brightness of the control region CR10 is lower than the predetermined brightness, the control unit 38 calculates a higher sensitivity than the present sensitivity of the imaging device 21. When the brightness of the control region CR10 is higher than the predetermined brightness, the control unit 38 calculates a lower sensitivity than the present sensitivity of the imaging device 21.

The control unit 38 generates a control signal indicating the calculated sensitivity and outputs the control signal to the imaging device 21. The imaging device 21 controls the sensitivity based on the control signal output from the control unit 38.

The control unit 38 may control either the amount of light of the light source 33 or the exposure amount of the imaging device 21. Alternatively, the control unit 38 may control both the amount of light of the light source 33 and the exposure amount of the imaging device 21.

The control region includes two or more pixels. The control unit 38 uses values of one or more pixels in the control region in Step S103.

After Step S103, the control unit 38 cuts out the control region from the image so as to generate a partial image and enlarges the partial image (Step S104).

The control unit 38 executes the following processing in Step S104. For example, the control unit 38 cuts out the control region CR10 shown in FIG. 7 from the image IMG10. The number of pixels of the control region CR10 is less than that of pixels of the image IMG10. The control unit 38 enlarges the control region CR10 such that the number of pixels of the control region CR10 matches the number of pixels of the image IMG10. By doing this, the control unit 38 generates a partial image. The partial image is larger than the control region CR10. In other words, the number of pixels of the partial image is greater than that of pixels of the control region CR10.

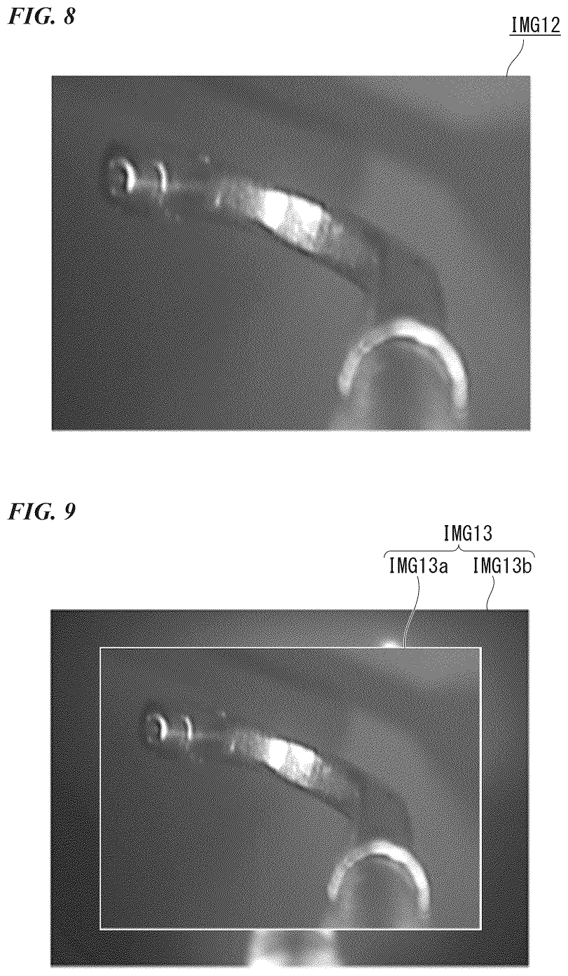

FIG. 8 shows an example of the partial image generated by the control unit 38. A partial image IMG12 is shown in FIG. 8. The partial image IMG12 is an enlarged image of the control region CR10 shown in FIG. 7. The number of pixels in the vertical direction of the partial image IMG12 is the same as that of pixels in the vertical direction of the image IMG10 shown in FIG. 3. The number of pixels in the horizontal direction of the partial image IMG12 is the same as that of pixels in the horizontal direction of the image IMG10 shown in FIG. 3.

After Step S104, the control unit 38 outputs the partial image to the display unit 36. By doing this, the control unit 38 displays the partial image on the display unit 36 (Step S105). For example, the control unit 38 displays the partial image IMG12 shown in FIG. 8 on the display unit 36. Accordingly, the enlarged control region is displayed on the display unit 36.

When the control unit 38 has determined that the treatment tool has not been detected in Step S101, the control unit 38 outputs the image output from the image-processing unit 30 to the display unit 36. By doing this, the control unit 38 displays the entire image generated by the imaging device 21 on the display unit 36 (Step S106).

When Step S105 or Step S106 has been executed, the image display processing shown in FIG. 5 is completed. The image display processing shown in FIG. 5 may be repeated.

For example, the imaging device 21 generates an image in each of two or more consecutive frame periods. The imaging device 21 generates a first image in a first frame period and generates a second image in a second frame period following the first frame period. An example of processing that uses the first image and the second image will be described.

To begin with, a first example will be described. The control unit 38 uses in Step S104 an image acquired before the exposure control is executed in Step S103. In such a case, the control unit 38 uses the same image in Step S100, Step S102, and Step S104. In other words, the control unit 38 uses a first image in Step S100, Step S102, and Step S104 and then uses a second image in Step S100, Step S102, and Step S104.

In the first example, after executing the exposure control by using values of pixels in a control region of the first image, the control unit 38 generates a partial image by using the first image. In addition, after executing the exposure control by using values of pixels in a control region of the second image, the control unit 38 generates a partial image by using the second image.

Next, a second example will be described. The control unit 38 uses in Step S104 an image acquired after the exposure control is executed in Step S103. In such a case, the control unit 38 executes Step S104 by using a different image from that used in Step S100 and Step S102. In other words, the control unit 38 uses a first image in Step S100 and Step S102 and then uses a second image in Step S104.

In the second example, after executing the exposure control by using values of pixels in a control region of the first image, the control unit 38 generates a partial image by using the second image.

In any one of the first and second examples, the exposure control is executed by using values of pixels in the control region of the first image. Therefore, the brightness of a region in which a treatment tool is seen in the second region is likely to be appropriate. Consequently, the visibility of the region is improved.

A control region in which the distal end of a treatment tool is seen is dark in many cases. Since the exposure control is executed based on the brightness of the control region in Step S103, the amount of light of the light source 33 or the exposure amount of the imaging device 21 increases in many cases. Therefore, halation is likely to occur in a region in which the base end of the treatment tool is seen.

The base end of the treatment tool is not seen in the partial image IMG12 shown in FIG. 8. Therefore, an influence of halation is suppressed in the partial image IMG12. The control unit 38 may display only the partial image IMG12 on the display unit 36. The control unit 38 need not display the region in which the base end of the treatment tool is seen on the display unit 36.

FIG. 9 shows another example of an image displayed on the display unit 36 in Step S105. An image IMG13 is shown in FIG. 9. The image IMG13 includes a partial image IMG13a and an image IMG13b. The partial image IMG13a is generated in Step S104. The image IMG13b is generated by the imaging device 21.

The control unit 38 superimposes the partial image IMG13a on the image IMG13b. Due to this, the partial image IMG13a overlaps part of the image IMG13b. The partial image IMG13a is displayed on the image IMG13b. Part of the image IMG13b is concealed by the partial image IMG13a. The size of the partial image IMG13a exceeds 50% of the size of the image IMG13b. The control unit 38 may superimpose the partial image IMG13a on a region of the image IMG13b in which the base end of a treatment tool is seen in order to suppress an influence of halation.

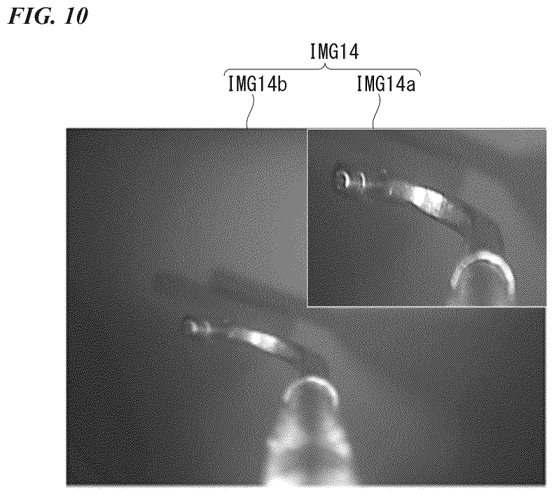

FIG. 10 shows another example of an image displayed on the display unit 36 in Step S105. An image IMG14 is shown in FIG. 10. The image IMG14 includes a partial image IMG14a and an image IMG14b. The partial image IMG14a is generated in Step S104. The image IMG14b is generated by the imaging device 21.

The control unit 38 superimposes the partial image IMG14a on the image IMG14b. Due to this, the partial image IMG14a overlaps part of the image IMG14b. The partial image IMG14a is displayed on the image IMG14b. Part of the image IMG14b is concealed by the partial image IMG14a. For example, the size of the partial image IMG14a is one-fourth that of the image IMG14b. The partial image IMG14a is superimposed on the upper right of the image IMG14b. When the halation has occurred in the image IMG14b and the visibility of the distal end of a treatment tool has deteriorated, a user can check the state of the distal end of the treatment tool in the partial image IMG14a.

In the examples shown in FIG. 9 and FIG. 10, the entire partial image overlaps part of the image generated by the imaging device 21. Part of the partial image may overlap part of the image generated by the imaging device 21.

The control unit 38 may display the image generated by the imaging device 21 and the partial image on the display unit 36 such that the image generated by the imaging device 21 and the partial image do not overlap each other.

The image-processing unit 30 may detect a region in which a treatment tool is seen in an image without executing the machine learning. For example, the memory 31 may store shape information indicating the shape of the treatment tool in advance. The image-processing unit 30 may detect a region having a shape that matches the shape indicated by the shape information from an image by executing pattern-matching processing. Alternatively, the image-processing unit 30 may detect a region in which a treatment tool is seen by detecting the edge of the treatment tool from an image.

The memory 31 may store region information indicating a relationship between the amount by which the treatment tool is pulled out of the distal end of the insertion unit 2, the position of a region of a treatment tool, and the size of the region in advance. The control unit 38 may determine the amount by which the treatment tool is pulled out of the distal end of the insertion unit 2 by determining the amount of movement of the treatment tool in the channel CH1. The image-processing unit 30 may extract a position and a size corresponding to the amount from the region information.

For example, the main body unit 3 includes a rotary encoder and two rollers. The two rollers are disposed so as to locate the channel CH1 therebetween. When a treatment tool is inserted into the channel CH1, the two rollers are in contact with the treatment tool. The two rollers rotate as the treatment tool moves. The rotary encoder determines the amount of rotation of at least one of the two rollers, thus determining a moving amount of the treatment tool. The rotary encoder outputs information indicating the determined moving amount. The control unit 38 may determine the amount by which the treatment tool is pulled out of the distal end of the insertion unit 2 based on the information.

As described above, the control unit 38 sets a region in which a treatment tool is seen as a control region. In addition to the region in which the treatment tool is seen, the control unit 38 may set a control region in light of another region. For example, in a case in which a treatment tool that holds an object is used, the control unit 38 may detect a first region in which the treatment tool is seen and a second region in which the object is seen. The control unit 38 may set a control region including the first region and the second region.

An operating method of an endoscope device according to each aspect of the present invention includes a determination step, a setting step, a control step, and a display step. The DL unit 300 determines, in the determination step (Step S100), the position and the size of a region including the distal end of a treatment tool seen in an image generated by the imaging device 21. The control unit 38 sets, in the setting step (Step S102), a control region in the image based on the position and the size determined in the determination step. The control unit 38 executes exposure control of controlling the brightness of the image by using a value of a pixel in the control region in the control step (Step S103). The control unit 38 displays the control region on the display unit 36 in the display step (Step S105).

Each aspect of the present invention may include the following modified example. The DL unit 300 learns the shape of a treatment tool by using an image generated by the imaging device 21. The DL unit 300 generates learning data indicating the features of the shape. The DL unit 300 determines the position and the size of the region including the distal end of the treatment tool by using the learning data.

Each aspect of the present invention may include the following modified example. The control unit 38 generates a partial image by cutting out the control region from the image generated by the imaging device 21. The control unit 38 displays the image generated by the imaging device 21 and the partial image on the display unit 36 such that at least part of the image generated by the imaging device 21 and at least part of the partial image overlap each other.

Each aspect of the present invention may include the following modified example. The control unit 38 generates a partial image by cutting out the control region from the image generated by the imaging device 21. The control unit 38 displays the image generated by the imaging device 21 and the partial image on the display unit 36 such that at least part of the image generated by the imaging device 21 and at least part of the partial image do not overlap each other.

Each aspect of the present invention may include the following modified example. The control unit 38 generates a partial image by cutting out the control region from the image generated by the imaging device 21. The control unit 38 displays an image generated by enlarging the partial image on the display unit 36.

In the first embodiment, the endoscope device 1 sets a control region in an image generated by the imaging device 21. In addition, the endoscope device 1 executes the exposure control by using values of pixels in the control region. Therefore, the endoscope device 1 can improve the visibility of an image in which a treatment tool is seen.

The endoscope device 1 displays an enlarged image of the control region. Therefore, the endoscope device 1 can improve the visibility of the control region in which the treatment tool is seen.

### Second Embodiment

A second embodiment of the present invention will be described. In the second embodiment, the endoscope device 1 shown in FIG. 1 is used. The endoscope device 1 performs edge enhancement in order to reduce the blurring of the distal end of a treatment tool in an image generated by the imaging device 21.

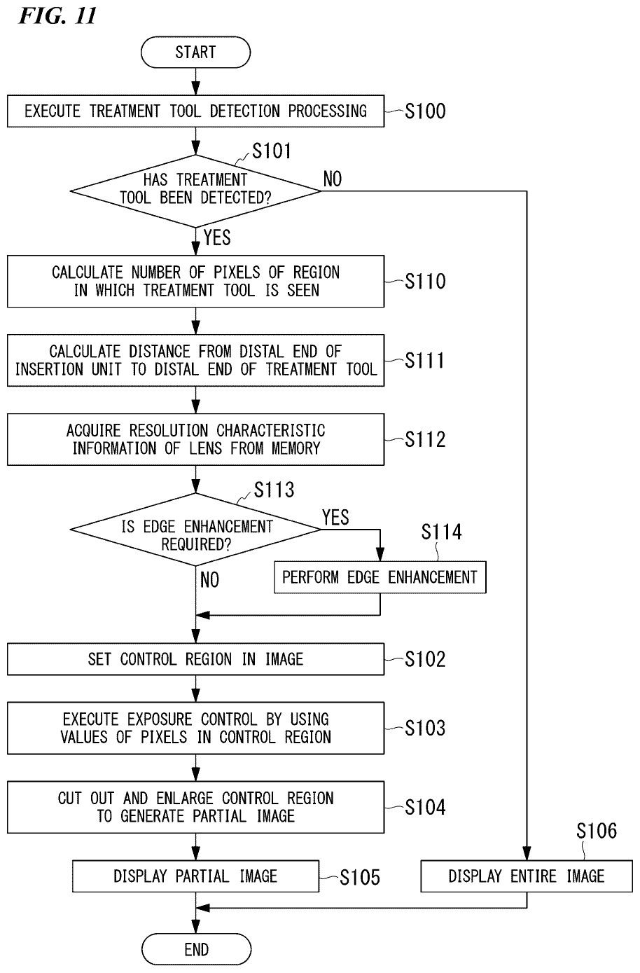

The endoscope device 1 executes image display processing shown in FIG. 11. FIG. 11 shows a procedure of the image display processing. The same processing as that shown in FIG. 5 will not be described.

When the control unit 38 has determined that the treatment tool has been detected in Step S101, the control unit 38 calculates the number of pixels of a region in which a treatment tool is seen in an image output from the image-processing unit 30 (Step S110).

After Step S110, the control unit 38 calculates the distance from the distal end of the insertion unit 2 to the distal end of the treatment tool (Step S111).

The control unit 38 executes the following processing in Step S111. As described above, the distal end of the treatment tool is seen at a position that is in accordance with the amount by which the treatment tool is pulled out of the distal end of the insertion unit 2. The distance between the distal end of the insertion unit 2 and the distal end of the treatment tool increases as the amount increases. The amount corresponds to the number of pixels calculated in Step S110. The distance and the number of pixels have a relationship that is in accordance with the type of the treatment tool.

The memory 35 stores distance information indicating a relationship between the distance and the number of pixels in advance. The distance information includes the distance and the number of pixels associated with each other. The distance information is prepared for each type of the treatment tool. The control unit 38 acquires the distance associated with the number of pixels calculated in Step S110 from the distance information.

After Step S111, the control unit 38 acquires the resolution characteristic information from the memory 35 (Step S112).

The resolution characteristic information indicates resolution characteristics of a lens group including the lens 40 and the lens 23. The lens 40 has resolution characteristics in accordance with the type of the optical adaptor 4. Therefore, the resolution characteristic information is prepared for each type of the optical adaptor 4. The memory 35 stores the resolution characteristic information in advance. The resolution characteristic information includes the distance and the resolution associated with each other.

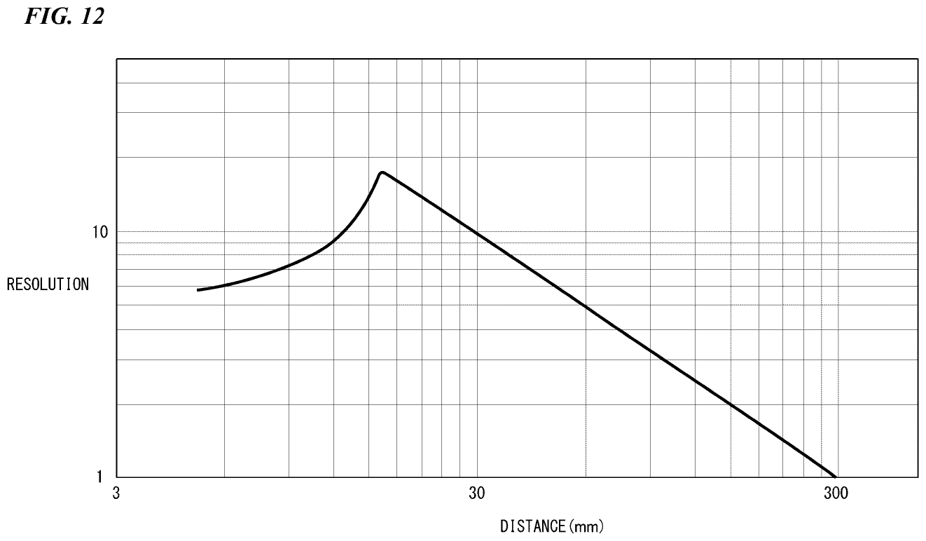

FIG. 12 shows an example of the resolution characteristic information. The resolution characteristic information is shown as a graph. The horizontal axis of the graph shown in FIG. 12 indicates the distance between the distal end of the insertion unit 2 and the distal end of the treatment tool. The vertical axis of the graph shown in FIG. 12 indicates the resolution. For example, the resolution is measured by using a USAF resolution test chart. The resolution increases as the value of the resolution shown in FIG. 12 increases.

When the resolution is high, the distal end of the treatment tool is unlikely to be blurred in an image generated by the imaging device 21. When the resolution is low, the distal end of the treatment tool is likely to be blurred in an image generated by the imaging device 21.

After Step S112, the control unit 38 determines the degree of blurring of the distal end of the treatment tool in an image based on the distance calculated in Step S11 and the resolution characteristic information acquired in Step S112. The control unit 38 determines whether the edge enhancement is required based on the degree of blurring (Step S113).

The control unit 38 executes the following processing in Step S113. The control unit 38 acquires the resolution associated with the distance calculated in Step S111 from the resolution characteristic information. The acquired resolution indicates the degree of blurring of the distal end of the treatment tool in the image. The control unit 38 determines whether the resolution is less than a predetermined value. When the resolution is less than the predetermined value, the control unit 38 determines that the edge enhancement is required. When the resolution is greater than or equal to the predetermined value, the control unit 38 determines that the edge enhancement is not required.

When the control unit 38 has determined that the edge enhancement is not required in Step S113, Step S102 is executed. When the control unit 38 has determined that the edge enhancement is required in Step S113, the control unit 38 performs the edge enhancement on the image output from the image-processing unit 30 (Step S114). After Step S114, Step S102 is executed.

The control unit 38 may determine the intensity of the edge enhancement in Step S114. For example, the control unit 38 may determine the intensity of the edge enhancement based on the resolution acquired from the resolution characteristic information and the above-described predetermined value. The control unit 38 may execute the edge enhancement by using a processing parameter that is in accordance with the intensity.

The control unit 38 may generate a control signal used for performing the edge enhancement and may output the control signal to the image-processing unit 30. The image-processing unit 30 may perform the edge enhancement based on the control signal.

In the image display processing shown in FIG. 11, the control region is set in an image after the edge enhancement is performed. A timing at which the edge enhancement is performed is not limited to that shown in FIG. 11. For example, after Step S104 is executed, Steps S110 to S114 may be executed. The control unit 38 may perform the edge enhancement on the partial image in Step S114. After Step S114 is executed, the control unit 38 may display the partial image on which the edge enhancement is performed on the display unit 36 by executing Step S105.

Each aspect of the present invention may include the following modified example. The control unit 38 determines the degree of blurring of the distal end of the insertion unit 2 by using a value of a pixel in the control region. The control unit 38 performs the edge enhancement on an image based on the degree.

In the second embodiment, the endoscope device 1 performs the edge enhancement on an image generated by the imaging device 21. By doing this, the endoscope device 1 can reduce the blurring of the distal end of a treatment tool in the image.

### Third Embodiment

A third embodiment of the present invention will be described. In the third embodiment, the endoscope device 1 shown in FIG. 1 is used.

When a treatment tool is detected in an image generated by the imaging device 21, the endoscope device 1 according to the first or second embodiment displays a partial image. On the other hand, the endoscope device 1 according to the third embodiment displays the entire image generated by the imaging device 21 regardless of whether a treatment tool is detected.

A control region in which the distal end of a treatment tool is seen is dark in many cases. In a case in which exposure control of controlling the amount of the illumination light in the light source 33 or the exposure amount of the imaging device 21 is applied to the entire image, the entire image is likely to be bright. Therefore, halation is likely to occur in a region in which the base end of a treatment tool is seen.

The image-processing unit 30 adjusts the brightness of the image generated by the imaging device 21. At this time, the image-processing unit 30 multiplies a value of each pixel of the image by a gain value. When the halation has occurred in the image generated by the imaging device 21, the control unit 38 controls the gain value by which the value of each pixel of the control region is multiplied. At this time, the control unit 38 increases the gain value such that the control region becomes bright.

The gain value can be set for each region of an image. The control unit 38 does not change the gain value by which the value of each pixel of a region other than the control region is multiplied. Therefore, the occurrence of the halation is prevented in a region in which the base end of a treatment tool is seen.

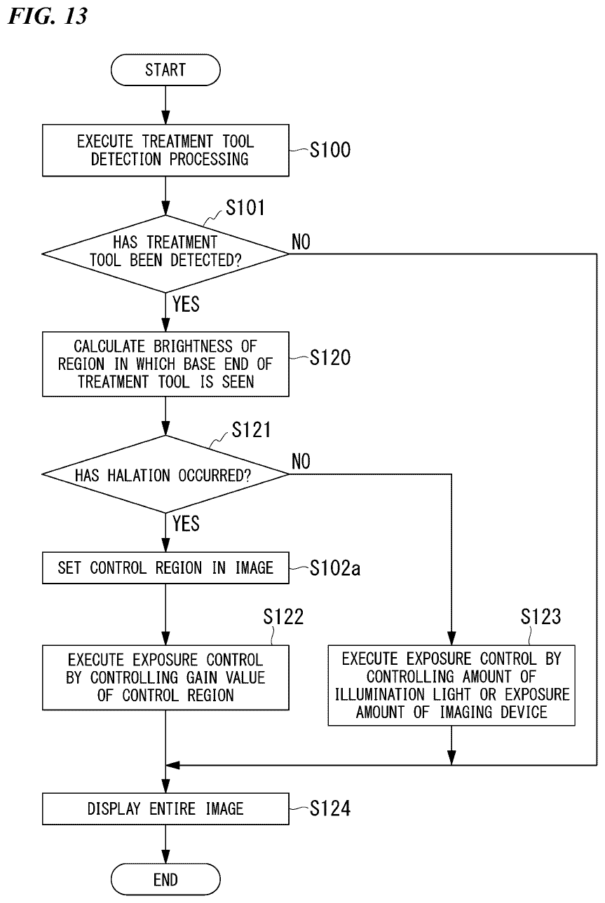

The endoscope device 1 executes image display processing shown in FIG. 13. FIG. 13 shows a procedure of the image display processing. The same processing as that shown in FIG. 5 will not be described.

When the control unit 38 has determined that the treatment tool has been detected in Step S101, the control unit 38 calculates the brightness of a region in which the base end of the treatment tool is seen based on values of pixels in the region (Step S120).

The treatment tool is inserted into the channel CH1 shown in FIG. 1. A positional relationship between the imaging device 21 and the channel CH1 is fixed. Therefore, the region in which the base end of the treatment tool is seen in the image generated by the imaging device 21 is fixed. The control unit 38 calculates the brightness of a region, which is set in advance, in Step S120.

After Step S120, the control unit 38 determines whether the brightness calculated in Step S120 is higher than predetermined brightness. By doing this, the control unit 38 determines whether halation has occurred in the region in which the base end of the treatment tool is seen (Step S121).

When the control unit 38 has determined that the brightness calculated in Step S120 is higher than the predetermined brightness in Step S121, the halation has occurred in the region in which the base end of the treatment tool is seen. In this case, the control unit 38 sets a control region in the image output from the image-processing unit 30 (Step S102a).

The control unit 38 sets a control region excluding the region in which the base end of the treatment tool is ween in Step S102a. By doing this, the control unit 38 sets a control region that excludes a region in which the halation has occurred.

Halation does not always occur in the entire region in which the base end of the treatment tool is seen. The control unit 38 may determine whether the halation has occurred in each pixel of the region in which the base end of the treatment tool is seen. The control unit 38 may set a control region excluding a region of pixels in which the halation has occurred. When the halation has occurred in part of the region in which the base end of the treatment tool is seen, the control region may include a region in which the base end of the treatment tool is seen and the halation has not occurred.

After Step S102a, the control unit 38 executes the exposure control by controlling a gain value by which the value of each pixel of the control region is multiplied (Step S122).

The control unit 38 executes the following processing in Step S122. The control unit 38 determines the brightness of the control region based on values of pixels in the control region. When the brightness of the control region is lower than the predetermined brightness, the control unit 38 calculates a gain value to increase the brightness of the control region to the predetermined brightness. When the brightness is higher than the predetermined brightness, the control unit 38 calculates a gain value to reduce the brightness of the control region to the predetermined brightness.

The control unit 38 generates a control signal indicating the calculated gain value and outputs the generated control signal to the image-processing unit 30. The image-processing unit 30 changes the gain value based on the control signal. At this time, the image-processing unit 30 changes only the gain value by which the value of each pixel of the control region is multiplied. The image-processing unit 30 multiplies the value of each pixel of the image generated by the imaging device 21 by the gain value. By doing this, the image-processing unit 30 adjusts the brightness of the image.

When the control unit 38 has determined that the brightness calculated in Step S120 is lower than the predetermined brightness in Step S121, the halation has not occurred in a range in which the base end of the treatment tool is seen. In this case, the control unit 38 executes the exposure control by controlling the amount of the illumination light in the light source 33 or the exposure amount of the imaging device 21 (Step S123).

For example, the control unit 38 determines the brightness of the image output from the image-processing unit 30 based on values of pixels in the entire image in Step S123. The control unit 38 controls the amount of light of the light source 33 or the exposure amount of the imaging device 21.

The control unit 38 need not use the entire image output from the image-processing unit 30 in Step S123. The control unit 38 may use a region that is larger than the control region set in Step S102a and is smaller than the entire image in order to determine the brightness of the image in Step S123. The region used for determining the brightness of the image in Step S123 may include the control region set in Step S102a. The control unit 38 may control both the amount of light of the light source 33 and the exposure amount of the imaging device 21 in Step S123.

After Step S122 or Step S123, the control unit 38 outputs the image processed in Step S122 or Step S123 to the display unit 36. By doing this, the control unit 38 displays the entire image on the display unit 36 (Step S124). When Step S124 has been executed, the image display processing shown in FIG. 13 is completed.

The image-processing unit 30 may adjust the brightness of the entire image generated by the imaging device 21 by using two or more types of gain values. Before or after Step S122, the control unit 38 may execute the exposure control by controlling a gain value used for adjusting the brightness of a region other than the control region. Halation has occurred in the region other than the control region. Therefore, the control unit 38 may control a gain value used for adjusting the brightness of the region in which the halation has occurred. The control unit 38 may reduce the brightness of the region to predetermined brightness by controlling the gain value.

Each aspect of the present invention may include the following modified example. The control unit 38 determines whether halation has occurred in an image generated by the imaging device 21. When the control unit 38 has determined that the halation has occurred, the control unit 38 executes the exposure control by using a value of a pixel in a control region.

Each aspect of the present invention may include the following modified example. When the control unit 38 has determined that the halation has occurred, the control unit 38 sets a control region that excludes a region in which the halation has occurred.

Each aspect of the present invention may include the following modified example. The image-processing unit 30 adjusts the brightness of an image generated by the imaging device 21 by using a gain value. When the control unit 38 has determined that the halation has occurred, the control unit 38 executes the exposure control by controlling the gain value used for adjusting the brightness of the control region.

Each aspect of the present invention may include the following modified example. When the control unit 38 has determined that the halation has not occurred, the control unit 38 executes the exposure control by using a value of a pixel in a region that includes the control region and is larger than the control region.

Each aspect of the present invention may include the following modified example. When the control unit 38 has determined that the halation has not occurred, the control unit 38 executes the exposure control by controlling at least one of the amount of the illumination light emitted to a treatment tool and the exposure amount of the imaging device 21.

When the control unit 38 has determined that the halation has occurred, the control unit 38 may execute processing in accordance with the size of a region in which the halation has occurred. When the size is smaller than a predetermined value, the control unit 38 may execute the exposure control by controlling at least one of the amount of the illumination light emitted to the treatment tool and the exposure amount of the imaging device 21. When the size is larger than the predetermined value, the control unit 38 may execute the exposure control by controlling a gain value.

In the third embodiment, the endoscope device 1 displays the entire image generated by the imaging device 21. When halation has occurred in the image, the endoscope device 1 adjusts the brightness of a control region. The control region excludes a region in which the halation has occurred. Therefore, the endoscope device 1 can improve the visibility of an image in which a treatment tool is seen and can suppress an influence of the halation.

### Fourth Embodiment

A fourth embodiment of the present invention will be described. In the fourth embodiment, the endoscope device 1 shown in FIG. 1 is used.

As described above, the halation is likely to occur in a region in which the base end of a treatment tool is seen. In addition, the halation is likely to occur in a region in which an object held by the treatment tool is seen. The endoscope device 1 determines whether the halation has occurred in the region in which the object is seen.

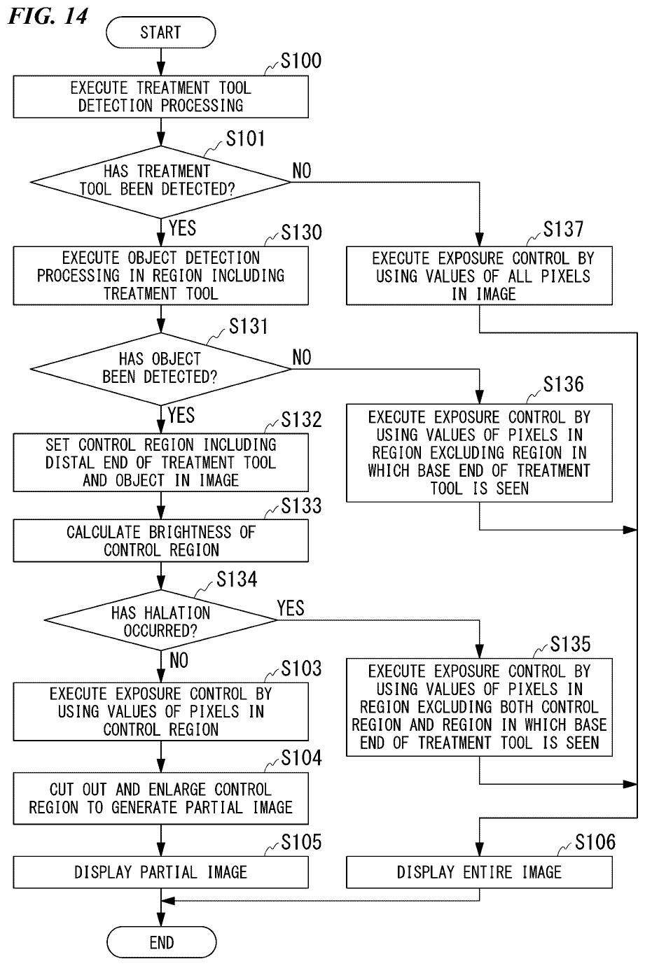

The endoscope device 1 executes image display processing shown in FIG. 14. FIG. 14 shows a procedure of the image display processing. The same processing as that shown in FIG. 5 will not be described.

When the control unit 38 has determined that the treatment tool has been detected in Step S101, the DL unit 300 executes object detection processing in a region including the detected treatment tool (Step S130).

Before the image display processing is executed, the DL unit 300 executes the following processing. The DL unit 300 executes machine learning that uses the image output from the imaging device 21 and learns the shape of an object held by a treatment tool seen in the image. For example, the DL unit 300 executes deep learning. The DL unit 300 generates learning data indicating the features of the shape of the object.

The DL unit 300 executes the following processing in Step S130. The DL unit 300 reads the learning data from the memory 31. The DL unit 300 executes the deep learning that uses the learning data and detects a region having similar features to those of the object indicated by the learning data. In other words, the DL unit 300 detects a region in the image in which the object is seen.

The DL unit 300 determines the position and the size of a region in which the object is seen and generates object position information and object size information. The object position information indicates the position of the object. The object size information indicates the size of the object. For example, the object position information and the object size information are integrated and indicate positions of two or more pixels included in the region in which the object is seen. The object position information may indicate the center position or the centroid position of the region. The object size information may indicate the number of pixels included in the region.

The DL unit 300 outputs the object position information and the object size information to the control unit 38. In a case in which a region in which the object is seen has not been detected, the DL unit 300 outputs information indicating that the object has not been detected to the control unit 38.

After Step S130, the control unit 38 determines whether an object has been detected based on the information output from the DL unit 300 (Step S131). When the object position information and the object size information are output from the DL unit 300, the control unit 38 determines that the object has been detected. When the information indicating that the object has not been detected is output from the DL unit 300, the control unit 38 determines that the object has not been detected.

When the control unit 38 has determined that the object has been detected in Step S131, the control unit 38 sets a control region in the image output from the image-processing unit 30. At this time, the control unit 38 sets the control region in the image based on the position information, the size information, the object position information, and the object size information. In other words, the control unit 38 sets, in the image, the control region that includes a region in which the distal end of the treatment tool and the object are seen and excludes a region in which the base end of the treatment tool is seen (Step S132).

After Step S132, the control unit 38 calculates the brightness of the control region based on values of pixels in the control region (Step S133).

After Step S133, the control unit 38 determines whether the brightness calculated in Step S133 is higher than predetermined brightness. By doing this, the control unit 38 determines whether halation has occurred in the control region (Step S134).

When the control unit 38 has determined that the brightness calculated in Step S133 is higher than the predetermined brightness in Step S134, the halation has occurred in the control region. In this case, the control unit 38 determines a region (base end region) in which the base end of the treatment tool is seen based on the position information and the size information. The control unit 38 executes the exposure control by using values of pixels in a new control region acquired by excluding the base end region and the control region set in Step S132 from the entire image output from the image-processing unit 30 (Step S135). The control unit 38 executes the exposure control without using values of pixels in the control region in which the halation has occurred and the base end region in which the halation is likely to occur. After Step S135, Step S106 is executed.

When the control unit 38 has determined that the brightness calculated in Step S133 is lower than the predetermined brightness in Step S134, the halation has not occurred in the control region. In this case, Step S103 is executed. The control unit 38 executes the exposure control by using values of pixels in the control region in Step S103.

When the control unit 38 has determined that the object has not been detected in Step S131, the control unit 38 determines a base end region based on the position information and the size information. The control unit 38 executes the exposure control by using values of pixels in a region (control region) acquired by excluding the base end region from the entire image output from the image-processing unit 30 (Step S136). The control unit 38 executes the exposure control without using values of pixels in the base end region in which the halation is likely to occur. After Step S136, Step S106 is executed.

When the control unit 38 has determined that the treatment tool has not been detected in Step S101, the control unit 38 executes the exposure control by using values of all the pixels in the image output from the image-processing unit 30 (Step S137). After Step S137, Step S106 is executed.

In the fourth embodiment, the endoscope device 1 executes the exposure control by using values of pixels in a region excluding the control region when the halation has occurred in the control region. The endoscope device 1 executes the exposure control by using values of pixels in the control region when the halation has not occurred in the control region. Therefore, the endoscope device 1 can improve the visibility of an image in which a treatment tool is seen and can suppress an influence of the halation.

### Fifth Embodiment

A fifth embodiment of the present invention will be described. In the fifth embodiment, the endoscope device 1 shown in FIG. 1 is used. The endoscope device 1 calculates the distance from the distal end of the insertion unit 2 to the distal end of a treatment tool and executes the exposure control based on the distance.

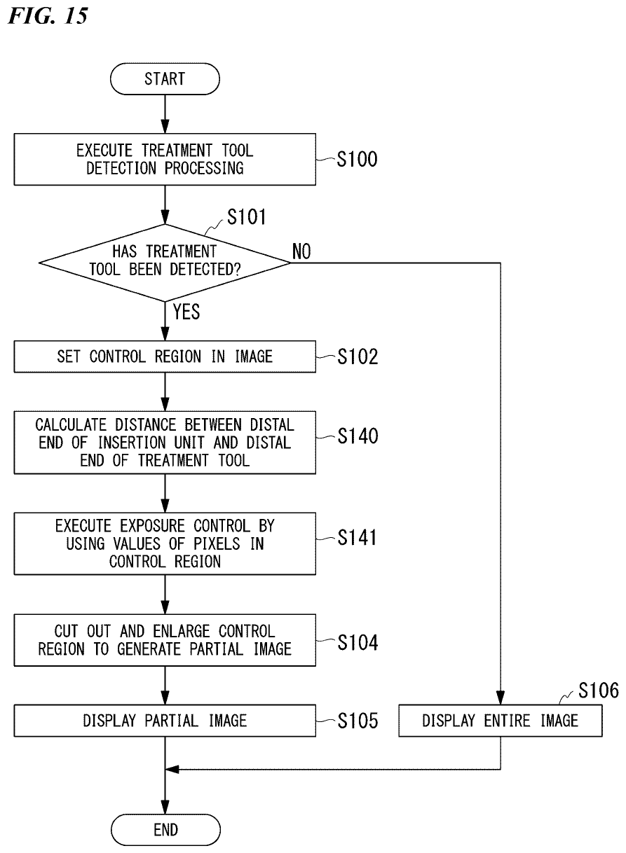

The endoscope device 1 executes image display processing shown in FIG. 15. FIG. 15 shows a procedure of the image display processing. The same processing as that shown in FIG. 5 will not be described.

After Step S102, the control unit 38 calculates the distance from the distal end of the insertion unit 2 to the distal end of a treatment tool by using values of pixels in the control region set in Step S102 (Step S140).

The control unit 38 executes the following processing in Step S140. The control unit 38 calculates the brightness of the treatment tool by using the values of the pixels in the control region. The control unit 38 calculates the amount of light incident on the imaging device 21 based on the brightness and a parameter of the imaging device 21. The parameter is an exposure time or the sensitivity.

The amount of light incident on the imaging device 21 changes in accordance with the amount of light of the light source 33, the reflectance of the treatment tool, the distance from the distal end of the insertion unit 2 to the distal end of the treatment tool, and the F value of each of the lenses 40 and 23. The reflectance and the F value are known. When the amount of light of the light source 33 is fixed, the amount of light incident on the imaging device 21 decreases as the distance increases. The control unit 38 calculates the distance based on the amount of light of the light source 33, the reflectance of the treatment tool, the F value of each of the lenses 40 and 23, and the amount of light incident on the imaging device 21.

After Step S140, the control unit 38 executes the exposure control by using the distance calculated in Step S140 (Step S141). After Step S141, Step S104 is executed.

The control unit 38 executes the following processing in Step S141. The brightness of an image decreases as the distance from the distal end of the insertion unit 2 to the distal end of the treatment tool increases. The memory 35 stores control information including the distance and a parameter of the exposure control in advance. The parameter is at least one of a setting value of the amount of light of the light source 33, the exposure time of the imaging device 21, and the sensitivity of the imaging device 21. The distance and the parameter are associated with each other in the control information.

The control unit 38 acquires a parameter associated with the distance calculated in Step S140 from the control information. The control unit 38 controls at least one of the amount of light of the light source 33 and the exposure amount of the imaging device 21 by using the acquired parameter.

The memory 35 may store distance information including the type of the optical adaptor 4, the size of a treatment tool, and the distance from the distal end of the insertion unit 2 to the distal end of the treatment tool in advance. The type, the size, and the distance are associated with each other in the distance information. The DL unit 300 may execute deep learning and may generate learning data indicating a relationship between the type, the size, and the distance. The learning data include the above-described distance information.

For example, a user inputs information indicating the type of the optical adaptor 4 into the endoscope device 1 by operating the operation unit 37. The control unit 38 may determine the type of the optical adaptor 4 based on the information.

When the optical adaptor 4 is mounted on the distal end portion 20, the optical adaptor 4 may be electrically connected to the control unit 38. The optical adaptor 4 may have a resistance value in accordance with the type of the optical adaptor 4. The control unit 38 may measure the resistance value and may determine the type of the optical adaptor 4 based on the resistance value.

As described above, the DL unit 300 outputs the position information indicating the position of the treatment tool and the size information indicating the size of the treatment tool to the control unit 38. The control unit 38 may acquire the distance information corresponding to the type of the optical adaptor 4 from the memory 35 and may determine the distance from the distal end of the insertion unit 2 to the distal end of the treatment tool based on the size information and the distance information. Specifically, the control unit 38 may acquire the distance associated with the size indicated by the size information from the distance information.

Each aspect of the present invention may include the following modified example. The imaging device 21 is disposed in the distal end portion 20 of the insertion unit 2 to be inserted inside an object. The optical adaptor 4 including the lens 40 is connected to the distal end portion 20. The control unit 38 determines the distance from the distal end portion 20 to the distal end of a treatment tool by using a value of a pixel in the control region. The control unit 38 executes the exposure control based on the distance.

Each aspect of the present invention may include the following modified example. The control unit 38 determines the distance from the distal end portion 20 to the distal end of the treatment tool based on the brightness of the treatment tool indicated by the value of the pixel in the control region.

Each aspect of the present invention may include the following modified example. The control unit 38 determines the type of the optical adaptor 4. The control unit 38 acquires distance information including the type of the optical adaptor 4, the size of the treatment tool, and the distance from the distal end portion 20 to the distal end of the treatment tool from the memory 35. The control unit 38 determines the distance from the distal end portion 20 to the distal end of the treatment tool based on the type of the optical adaptor 4 determined by the control unit 38, the size of the treatment tool determined by the DL unit 300, and the distance information.

In the fifth embodiment, the endoscope device 1 executes the exposure control based on the distance from the distal end of the insertion unit 2 to the distal end of a treatment tool. Therefore, the endoscope device 1 can improve the visibility of an image in which the treatment tool is seen.

While preferred embodiments of the invention have been described and shown above, it should be understood that these are examples of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An endoscope device, comprising:
an imaging device configured to generate an image in which a treatment tool is seen;
a determination unit configured to determine a position and a size of a region including a distal end of the treatment tool seen in the image; and
a control unit configured to:
set a control region in the image based on the position and the size determined by the determination unit;
execute exposure control of controlling brightness of the image by using a value of a pixel in the control region; and
display, on a display, a control region image including the control region after the exposure control is executed,
wherein the control region includes a first region and excludes a second region,
wherein the distal end is seen in the first region,
wherein a base end of the treatment tool is seen in the second region, and
wherein the second region is in contact with a boundary of the image.

2. The endoscope device according to claim 1,
wherein the control unit is configured to:
determine whether halation has occurred in the image; and
execute the exposure control when the control unit has determined that the halation has occurred.

3. The endoscope device according to claim 2,
wherein the control unit is configured to set the control region that excludes a region in which the halation has occurred when the control unit has determined that the halation has occurred.

4. The endoscope device according to claim 2, further comprising an image-processing circuit configured to adjust brightness of the image by using a gain value,
wherein the control unit is configured to execute the exposure control by controlling the gain value used for adjusting brightness of the control region when the control unit has determined that the halation has occurred.

5. The endoscope device according to claim 2,
wherein the control unit is configured to execute the exposure control by using a value of a pixel in a region that includes the control region and is larger than the control region when the control unit has determined that the halation has not occurred.

6. The endoscope device according to claim 5,
wherein the control unit is configured to execute the exposure control by controlling at least one of an amount of illumination light emitted to the treatment tool and an exposure amount of the imaging device when the control unit has determined that the halation has not occurred.

7. The endoscope device according to claim 1,
wherein the determination unit is configured to:
learn a shape of the treatment tool by using the image;
generate learning data indicating features of the shape; and
determine the position and the size by using the learning data.

8. The endoscope device according to claim 1,
wherein the imaging device is disposed in a distal end portion of an insertion unit to be inserted inside an object,
wherein an optical adaptor including a lens is connected to the distal end portion, and
wherein the control unit is configured to:
determine a distance from the distal end portion to the distal end of the treatment tool by using the value of the pixel; and
execute the exposure control based on the distance.

9. The endoscope device according to claim 8,
wherein the control unit is configured to determine the distance based on brightness of the treatment tool indicated by the value of the pixel.

10. The endoscope device according to claim 8,
wherein the control unit is configured to:
determine a type of the optical adaptor;
acquire, from a memory, distance information including a type of the optical adaptor, a size of the treatment tool, and the distance; and
determine the distance based on the type determined by the control unit, the size determined by the determination unit, and the distance information.

11. The endoscope device according to claim 1,
wherein the control unit is configured to:
generate a partial image by cutting out the control region from the image generated by the imaging device; and
display the image generated by the imaging device and the partial image on the display such that at least part of the image generated by the imaging device and at least part of the partial image overlap each other.

12. The endoscope device according to claim 1,
wherein the control unit is configured to:
generate a partial image by cutting out the control region from the image generated by the imaging device; and
display the image generated by the imaging device and the partial image on the display such that the image generated by the imaging device and the partial image do not overlap each other.

13. The endoscope device according to claim 1,
wherein the control unit is configured to:
generate a partial image by cutting out the control region from the image generated by the imaging device; and
display an image generated by enlarging the partial image on the display.

14. The endoscope device according to claim 1,
wherein the control unit is configured to:
determine a degree of blurring of the distal end by using the value of the pixel; and
perform edge enhancement on the image based on the degree.

15. A method of operating an endoscope device including an imaging device configured to generate an image in which a treatment tool is seen, the method comprising:
determining a position and a size of a region including a distal end of the treatment tool seen in the image;
setting a control region in the image based on the position and the size;
executing exposure control of controlling brightness of the image by using a value of a pixel in the control region; and
displaying, on a display, a control region image including the control region after the exposure control is executed,
wherein the control region includes a first region and excludes a second region,
wherein the distal end is seen in the first region,
wherein a base end of the treatment tool is seen in the second region, and
wherein the second region is in contact with a boundary of the image.

16. A non-transitory computer-readable recording medium storing a program causing a computer of an endoscope device including an imaging device configured to generate an image in which a treatment tool is seen to execute:
determining a position and a size of a region including a distal end of the treatment tool seen in the image;
setting a control region in the image based on the position and the size;
executing exposure control of controlling brightness of the image by using a value of a pixel in the control region; and
displaying, on a display, a control region image including the control region after the exposure control is executed,
wherein the control region includes a first region and excludes a second region,
wherein the distal end is seen in the first region,
wherein a base end of the treatment tool is seen in the second region, and
wherein the second region is in contact with a boundary of the image.

17. An endoscope device, comprising:
an imaging device configured to generate an image in which a treatment tool is seen; and
a processor configured to:
determine a position and a size of a region including a distal end of the treatment tool seen in the image;
set a control region in the image based on the position and the size;
execute exposure control of controlling brightness of the image by using a value of a pixel in the control region; and
display, on a display, a control region image including the control region after the exposure control is executed,
wherein the control region includes a first region and excludes a second region,
wherein the distal end is seen in the first region,
wherein a base end of the treatment tool is seen in the second region, and
wherein the second region is in contact with a boundary of the image.
